SEQUENCE LISTING (12 sequences)

SEQ ID NO: 1 - DNA, 93 bp, artificial sequence
  DNA sequence encoding the SP17 signal peptide
atgcatcttgctatcaagtctctctttgtctctctcctcggagccagcgttctcgcaagccctcttcccagcaatgctctggttgagagagcc

SEQ ID NO: 2 - protein, 31 aa, artificial sequence
  The SP17 signal peptide
MHLAIKSLFVSLLGASVLASPLPSNALVERA

SEQ ID NO: 3 - DNA, 57 bp, artificial sequence
  DNA sequence encoding the SP20 signal peptide
atgcgctggaagttcaccctctccttcctcctcctcctctccggccgcgccctcgcc

SEQ ID NO: 4 - protein, 19 aa, artificial sequence
  The SP20 signal peptide
MRWKFTLSFLLLLSGRALA

SEQ ID NO: 5 - DNA, 93 bp, Thermomyces lanuginosus
atggtcggctttacccccgttgcccttgcggccttagccgcgactggggccctggccttcccggcagggaatgccacggagctcgaaaagcga

SEQ ID NO: 6 - protein, 31 aa, Thermomyces lanuginosus
MVGFTPVALAALAATGALAFPAGNATELEKR

SEQ ID NO: 7 - DNA, 582 bp, Thermomyces lanuginosus
cagacaacccccaactcggagggctggcacgatggttattactattcctggtggagtgacggtggagcgcaggccacgtacaccaacctggaaggcggcacctacgagatcagctggggagatggcggtaacctcgtcggtggaaagggctggaaccccggcctgaacgcaagagccatccactttgagggtgtttaccagccaaacggcaacagctaccttgcggtctacggttggacccgcaacccgctggtcgagtattacatcgtcgagaactttggcacctatgatccttcctccggtgctaccgatctaggaactgtcgagtgcgacggtagcatctatcgactcggcaagaccactcgcgtcaacgcacctagcatcgacggcacccaaaccttcgaccaatactggtcggtccgccaggacaagcgcaccagcggtaccgtccagacgggctgccacttcgacgcctgggctcgcgctggtttgaatgtcaacggtgaccactactaccagatcgttgcaacggagggctacttcagcagcggctatgctcgcatcaccgttgctgacgtgggc

SEQ ID NO: 8 - protein, 194 aa, Thermomyces lanuginosus
QTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 9 - DNA, 7005 bp, artificial sequence
  Plasmid pJaL537; figure 6.
aattcatggtgttttgatcattttaaatttttatatggcgggtggtgggcaactcgcttgcgcgggcaactcgcttaccgattacgttagggctgatatttacgtaaaaatcgtcaagggatgcaagaccaaagtactaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactggggatccaccatggtcggctttacccccgttgcccttgcggccttagccgcgactggggccctggccttcccggcagggaatgccacggagctcgaaaagcgacagacaacccccaactcggagggctggcacgatggttattactattcctggtggagtgacggtggagcgcaggccacgtacaccaacctggaaggcggcacctacgagatcagctggggagatggcggtaacctcgtcggtggaaagggctggaaccccggcctgaacgcaagagccatccactttgagggtgtttaccagccaaacggcaacagctaccttgcggtctacggttggacccgcaacccgctggtcgagtattacatcgtcgagaactttggcacctatgatccttcctccggtgctaccgatctaggaactgtcgagtgcgacggtagcatctatcgactcggcaagaccactcgcgtcaacgcacctagcatcgacggcacccaaaccttcgaccaatactggtcggtccgccaggacaagcgcaccagcggtaccgtccagacgggctgccacttcgacgcctgggctcgcgctggtttgaatgtcaacggtgaccactactaccagatcgttgcaacggagggctacttcagcagcggctatgctcgcatcaccgttgctgacgtgggctaactcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcctctagactggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatacccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagatctagagtcgacctgcaggcatgcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctattttcaattcaattcatcattttttttttattcttctttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccatg

SEQ ID NO: 10 - DNA, 10232 bp, artificial sequence
  Plasmid pAUT751; figure 7.
gagaggaaaaggaagaagatggtggggttcagaaggaggggttgagttaaatagcatgggttgagtcaacgtgataagggcactataccgtatagatcagcggcacccgattctatccgttccttttgctcctctttagctttgaccggtgagccggacaagaaacaagtgaaatcatcctgacatcggcggacgatctcctagcttttacatttcgttaccaatgggatcccgtaatcaattgcccgtctgtcagatcgaggaatggtggatcatctgatccactatataacatagtactctgtccacggttagttaacatttatccaaaaacagaaaatctccacgactggaaccttttcaatagtgaaacctaagtattagcaaataatcgttacccccactcgcactccataatccttgatccagttctttttctttccgactttctcgactttctccgccctttcctgtaagctgtcaagagagaacgcggcgacagccacatcgttgttgcatttcactttgagacgcagcatatcgcactcgtctttcttcttcacaatggattcccattcagtttccaatcggatcctccaatgcatgtccgacacgcggccggatggatgcacgtctccaattagggtcgctacccgacccaagtactgacaccaccatgcctcctgcttagagacacaccggctcaattgccggtgaaattcttcatcggtgggtgcaaggtcccctgcccagcgctcacagagtatcaaggcaatcagggcacgttcggtgtgtgatagtgtatttaccgaaggcgcgccccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactaccatggtcggctttacccccgttgcccttgcggccttagccgcgactggggccctggccttcccggcagggaatgccacggagctcgaaaagcgacagacaacccccaactcggagggctggcacgatggttattactattcctggtggagtgacggtggagcgcaggccacgtacaccaacctggaaggcggcacctacgagatcagctggggagatggcggtaacctcgtcggtggaaagggctggaaccccggcctgaacgcaagagccatccactttgagggtgtttaccagccaaacggcaacagctaccttgcggtctacggttggacccgcaacccgctggtcgagtattacatcgtcgagaactttggcacctatgatccttcctccggtgctaccgatctaggaactgtcgagtgcgacggtagcatctatcgactcggcaagaccactcgcgtcaacgcacctagcatcgacggcacccaaaccttcgaccaatactggtcggtccgccaggacaagcgcaccagcggtaccgtccagacgggctgccacttcgacgcctgggctcgcgctggtttgaatgtcaacggtgaccactactaccagatcgttgcaacggagggctacttcagcagcggctatgctcgcatcaccgttgctgacgtgggctaactcgagaatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaattggcggtgatattgatggcacgatagaagcagcacattctgtccctagacttagagattatcatgaagatattctcgatcaaatgctttttgcgctctttctcagcaaaagacatgctgatgcagcgaagctgctggaaagtattttcggtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatcccaatttagaagaagtgaaggtctgattctttccatccttccgccaacagtatcctccgagccgattcttccatggctggcggaccacaaatcaggaccatactctcatcttctggagccgcgtactcctttaggagctcttcggatatgcgtcctcggcggccagtccatgagtccggcgctttggatagggtgtgtattatattacaccttctgctgtcggttgccatgaagccgtcgagctcagcccggcaaaggatatcttcctcctgtctgtttccattgaggactgtacaagaggtgggatcttgccggtcctgaaccacggcgcgcaagacctggaagatcggtgtgataccggttcctccacaaatcatcttaaacgaccgaacatggcgttccttcccacttatgacaactcgtccatttccaaggtattcgaatctgcctgtcggacccttgcattccaccacggagcccaatggcagcctatccagggccatcgtcatcttgccgcctgccgaggtggctgttgcaaagtatactttaaccagcaagtccacggtccctttctggctggtttcagaaattggggtgtatgagcggatgatggcttcgttgttggatgatgtgtcgaggactttgatcataagatgctggccgactggtaaacccaatgtttgatcttcgtgttccaatttgaaactaaatattcgtgtatcccaggatatgtctttcctttctttcaatgttgcctttgtccaagaccgtgattggaggaacactgggcgaatttcatcggtggaggatgatgcatcatccttgagtgcttttaaaccttccgggtccatcgttccaatatggtactcaggcatcatcgcctttgccgtctcgctatctatggataggtgtcaatagatggtacaattgcagtgtgatatttttgggactcacgaatagcaaggaattcctcagagacatccagaccagcagaggagataatactctgcgctccgccagggtggccttcaagaaatgcttgaccatcatacacttctccattcacgatgaaccatggcttctcatcgcaggaattctccttgaattcttcaaaaccaatcactcggcttagcccgtctttcttcatattaatgtcttgcacgggctccggctccgtcggctcctctccttcgtgtctttctccccagttaccattcgtcaggtcacccccagcctttttgacgcgttccatccatcctgtaggcatactagggtgggtagggtgctcgaatctcaagttcccgttttccttcgtaattgtaacccggaaccacgggttgttcatcattccgagaacggaccagtacatatcgcgaggctgcacgcccaatgcttcgtccatggctcttacaaggatggcatcactgttctcaagctctgggatggtgatgcttagagaccaaaaacaccagcagaagcaagtttcgcgccagtacatatctactttgcctccaaaaagctcgccttcaaaatcacgatacttgtcttcggcatattcgatttccgccaatctccaagctataagtccgttagctttgataagcattctcacacatcgagcgagcgagggtgcgtacatttgcctttgtctagggatatttctaccctggtaaccctgcggcccccaccggcgtatgcatatcctctgacagtatatgacggccctgccgacaggagatttaagacctcattgttttggggatatgcaacggcggagttggtgtttaggtcataaatcgcataccgctcatcgtgccaccaatttcggttatttgatgccatctcaggcgagaccattgttctgggttagggagttagacaaatgatggaaatataaaataagtgccctttagacatacggtaagacgcggttgtcattgatatggtaccagttgtcgcttggtgcatcggtcaagatcagcctcttcagccacttaacacttcgtcctcctatttgaccgggcacgacggccctcagcggacgaccatgatctgggcgaagagactccccgttcattttatgtgcaagcatgatccccctgttggggtccagggcccagttcaatttaatagatgtgccgtagtgaccattgggctgcggcgaacttagcaattatcatcataagatagaggtacagcataccagcttatccgctccttccatacagacgtatttcgctttacgcaggggtttcgcactgcggagaatatccgccagcaatgggccagtgaagagggcagtcgatagtcccgccgatccccaggaaaaacctttcgttttacgtacaatgttttgctctttgcgtcgattgccagcacatacgagggtgataggcgctgttatttggtcgtactgctgcaacacttgtcggaagtttagtaccaaaggcttctctaccagtctatactttggttaacggatgtttggcagagaacctagcactatactaacccttcgatgctaatttcccagtgagggatatcttcatccttgatatgagggactgggccatgatttcgaacatagaagagctccggcgatgttaaaaaccctttcagagtgtgagaatgtaacggctcaaggggacaagcatgacagccggtgcaagcaacctgataaggataggagtggagcagttataactcataccttctttatacagatctcgagctcgcgaaagcttactagtgcatgcgtttctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccgacaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagactagtgcatgccctagggtcgacttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactgccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatactcttcctttttcaatattattgaagcatttatcagggttattgtctcatgagcggatacatatttgaatgtataaactagtgcatgcaagcttattagtgataccccactctaagaaaatagaccaatctccagctgcaccttcagacactccggtacaaattctcgtctatgttggagattgttgtgactttgaaacatgacccttgaccctgattttgaatttgtccatatatcgaggcaggtgtcttattcgtacggagagggtatctgtcgtagacacatagtagtagtcatttcgagtgctgaatttataaatcgcatcatacttgcgacatactgccataaaaggagtacgtatccaccactacttattgcgcaccaacacgcttcaggtatgcatcccatccctccttctggtactgcttcgccgcctccacgggatcaggagcagcataaattccacggccagcaataataaagtcggcaccgcgtccaacagccgactcaggagtttggtactgctgtcccagcttgtcacccttcgaggagaggttgacacctgtcgtgaagacgacaaaatcttcctcctccgaaggcgagctaacttcagactgaacctcgccaaggtgacgtgtcgagacgaatcccatcacaaacttcttatacttccgagcatagtcaacagaagaagtagtatattgaccggtagccaaagatcccttggaggtcatctccgcaaggatcaaaaggcccctctcggagccgtaggggaagtcctcggccgaagcagtctgggccagagcctcgacgataccctcaccgggcagaatactgcagttgatgatgtgggcccactcagagatacgcagagtgccgccatggtactgcttttggactgtgtttccgatatcgatgaacttgcgatcttcgaagatgaggaaattgtgcttctctgcaagggccttcagaccggtgatggtttcttcgctgaaatcggagaggatatcgatgtgagttttgatcacggcaatgtacggaccgagtcctgttatataatccaccattaaccattactagatcacatgtaagtggcatccccggtgcgcatacggtcagccaaatccagcagctctttggtggttgtcacgtcggcggaaacggtgacattggttttcttggcctcggcaacctcgaagagcttctttacgagcgcattggggtgcttgctagcgcgtgcgctgtaggtcaattgcgacttggaagacatggtgccgcggcaatgaggatcatctgttagccattccatcaacaggaagaacgagagaaggcatgatccttttcgctggtattatccagatcaagttttagccgtataatctcagaacgaacccagtccatcgatgccatgtccttctagactaggatcctagagtctagggcccagcttagggagggcatgtgaatgcatcgatgactgggaacgaacaccggcccacgccaaagacgttacctaagataccttgatcattgtgagagtccagccaaaagtattccatgacttccatcgtatgccctctagagggctaatcgaggagtgtatttacattgtcggttggtttgggaactatagaagatggtcagttattccaatcaccaaaggtttatcgaagggaggaagacttgttcagtttcgtccgaggacttttggaattcaaatctgagatagagaattgtgtgggat

SEQ ID NO: 11 - DNA, 10271 bp, artificial sequence
  Plasmid pAUT654; figure 8.
gagaggaaaaggaagaagatggtggggttcagaaggaggggttgagttaaatagcatgggttgagtcaacgtgataagggcactataccgtatagatcagcggcacccgattctatccgttccttttgctcctctttagctttgaccggtgagccggacaagaaacaagtgaaatcatcctgacatcggcggacgatctcctagcttttacatttcgttaccaatgggatcccgtaatcaattgcccgtctgtcagatcgaggaatggtggatcatctgatccactatataacatagtactctgtccacggttagttaacatttatccaaaaacagaaaatctccacgactggaaccttttcaatagtgaaacctaagtattagcaaataatcgttacccccactcgcactccataatccttgatccagttctttttctttccgactttctcgactttctccgccctttcctgtaagctgtcaagagagaacgcggcgacagccacatcgttgttgcatttcactttgagacgcagcatatcgcactcgtctttcttcttcacaatggattcccattcagtttccaatcggatcctccaatgcatgtccgacacgcggccggatggatgcacgtctccaattagggtcgctacccgacccaagtactgacaccaccatgcctcctgcttagagacacaccggctcaattgccggtgaaattcttcatcggtgggtgcaaggtcccctgcccagcgctcacagagtatcaaggcaatcagggcacgttcggtgtgtgatagtgtatttaccgaaggcgcgccccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactaccatgcatcttgctatcaagtctctctttgtctctctcctcggagccagcgttctcgcaagccctcttcccagcaatgctctggttgagagagccttcccggcagggaatgccacggagctcgaaaagcgacagacaacccccaactcggagggctggcacgatggttattactattcctggtggagtgacggtggagcgcaggccacgtacaccaacctggaaggcggcacctacgagatcagctggggagatggcggtaacctcgtcggtggaaagggctggaaccccggcctgaacgcaagagccatccactttgagggtgtttaccagccaaacggcaacagctaccttgcggtctacggttggacccgcaacccgctggtcgagtattacatcgtcgagaactttggcacctatgatccttcctccggtgctaccgatctaggaactgtcgagtgcgacggtagcatctatcgactcggcaagaccactcgcgtcaacgcacctagcatcgacggcacccaaaccttcgaccaatactggtcggtccgccaggacaagcgcaccagcggtaccgtccagacgggctgccacttcgacgcctgggctcgcgctggtttgaatgtcaacggtgaccactactaccagatcgttgcaacggagggctacttcagcagcggctatgctcgcatcaccgttgctgacgtgggctaactcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaattggcggtgatattgatggcacgatagaagcagcacattctgtccctagacttagagattatcatgaagatattctcgatcaaatgctttttgcgctctttctcagcaaaagacatgctgatgcagcgaagctgctggaaagtattttcggtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatcccaatttagaagaagtgaaggtctgattctttccatccttccgccaacagtatcctccgagccgattcttccatggctggcggaccacaaatcaggaccatactctcatcttctggagccgcgtactcctttaggagctcttcggatatgcgtcctcggcggccagtccatgagtccggcgctttggatagggtgtgtattatattacaccttctgctgtcggttgccatgaagccgtcgagctcagcccggcaaaggatatcttcctcctgtctgtttccattgaggactgtacaagaggtgggatcttgccggtcctgaaccacggcgcgcaagacctggaagatcggtgtgataccggttcctccacaaatcatcttaaacgaccgaacatggcgttccttcccacttatgacaactcgtccatttccaaggtattcgaatctgcctgtcggacccttgcattccaccacggagcccaatggcagcctatccagggccatcgtcatcttgccgcctgccgaggtggctgttgcaaagtatactttaaccagcaagtccacggtccctttctggctggtttcagaaattggggtgtatgagcggatgatggcttcgttgttggatgatgtgtcgaggactttgatcataagatgctggccgactggtaaacccaatgtttgatcttcgtgttccaatttgaaactaaatattcgtgtatcccaggatatgtctttcctttctttcaatgttgcctttgtccaagaccgtgattggaggaacactgggcgaatttcatcggtggaggatgatgcatcatccttgagtgcttttaaaccttccgggtccatcgttccaatatggtactcaggcatcatcgcctttgccgtctcgctatctatggataggtgtcaatagatggtacaattgcagtgtgatatttttgggactcacgaatagcaaggaattcctcagagacatccagaccagcagaggagataatactctgcgctccgccagggtggccttcaagaaatgcttgaccatcatacacttctccattcacgatgaaccatggcttctcatcgcaggaattctccttgaattcttcaaaaccaatcactcggcttagcccgtctttcttcatattaatgtcttgcacgggctccggctccgtcggctcctctccttcgtgtctttctccccagttaccattcgtcaggtcacccccagcctttttgacgcgttccatccatcctgtaggcatactagggtgggtagggtgctcgaatctcaagttcccgttttccttcgtaattgtaacccggaaccacgggttgttcatcattccgagaacggaccagtacatatcgcgaggctgcacgcccaatgcttcgtccatggctcttacaaggatggcatcactgttctcaagctctgggatggtgatgcttagagaccaaaaacaccagcagaagcaagtttcgcgccagtacatatctactttgcctccaaaaagctcgccttcaaaatcacgatacttgtcttcggcatattcgatttccgccaatctccaagctataagtccgttagctttgataagcattctcacacatcgagcgagcgagggtgcgtacatttgcctttgtctagggatatttctaccctggtaaccctgcggcccccaccggcgtatgcatatcctctgacagtatatgacggccctgccgacaggagatttaagacctcattgttttggggatatgcaacggcggagttggtgtttaggtcataaatcgcataccgctcatcgtgccaccaatttcggttatttgatgccatctcaggcgagaccattgttctgggttagggagttagacaaatgatggaaatataaaataagtgccctttagacatacggtaagacgcggttgtcattgatatggtaccagttgtcgcttggtgcatcggtcaagatcagcctcttcagccacttaacacttcgtcctcctatttgaccgggcacgacggccctcagcggacgaccatgatctgggcgaagagactccccgttcattttatgtgcaagcatgatccccctgttggggtccagggcccagttcaatttaatagatgtgccgtagtgaccattgggctgcggcgaacttagcaattatcatcataagatagaggtacagcataccagcttatccgctccttccatacagacgtatttcgctttacgcaggggtttcgcactgcggagaatatccgccagcaatgggccagtgaagagggcagtcgatagtcccgccgatccccaggaaaaacctttcgttttacgtacaatgttttgctctttgcgtcgattgccagcacatacgagggtgataggcgctgttatttggtcgtactgctgcaacacttgtcggaagtttagtaccaaaggcttctctaccagtctatactttggttaacggatgtttggcagagaacctagcactatactaacccttcgatgctaatttcccagtgagggatatcttcatccttgatatgagggactgggccatgatttcgaacatagaagagctccggcgatgttaaaaaccctttcagagtgtgagaatgtaacggctcaaggggacaagcatgacagccggtgcaagcaacctgataaggataggagtggagcagttataactcataccttctttatacagatctcgatcgagctcgcgaaagcttactagtgcatgcgtttctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccgacaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagactagtgcatgccctagggtcgacttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactgccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatactcttcctttttcaatattattgaagcatttatcagggttattgtctcatgagcggatacatatttgaatgtataaactagtgcatgcaagcttattagtgataccccactctaagaaaatagaccaatctccagctgcaccttcagacactccggtacaaattctcgtctatgttggagattgttgtgactttgaaacatgacccttgaccctgattttgaatttgtccatatatcgaggcaggtgtcttattcgtacggagagggtatctgtcgtagacacatagtagtagtcatttcgagtgctgaatttataaatcgcatcatacttgcgacatactgccataaaaggagtacgtatccaccactacttattgcgcaccaacacgcttcaggtatgcatcccatccctccttctggtactgcttcgccgcctccacgggatcaggagcagcataaattccacggccagcaataataaagtcggcaccgcgtccaacagccgactcaggagtttggtactgctgtcccagcttgtcacccttcgaggagaggttgacacctgtcgtgaagacgacaaaatcttcctcctccgaaggcgagctaacttcagactgaacctcgccaaggtgacgtgtcgagacgaatcccatcacaaacttcttatacttccgagcatagtcaacagaagaagtagtatattgaccggtagccaaagatcccttggaggtcatctccgcaaggatcaaaaggcccctctcggagccgtaggggaagtcctcggccgaagcagtctgggccagagcctcgacgataccctcaccgggcagaatactgcagttgatgatgtgggcccactcagagatacgcagagtgccgccatggtactgcttttggactgtgtttccgatatcgatgaacttgcgatcttcgaagatgaggaaattgtgcttctctgcaagggccttcagaccggtgatggtttcttcgctgaaatcggagaggatatcgatgtgagttttgatcacggcaatgtacggaccgagtcctgttatataatccaccattaaccattactagatcacatgtaagtggcatccccggtgcgcatacggtcagccaaatccagcagctctttggtggttgtcacgtcggcggaaacggtgacattggttttcttggcctcggcaacctcgaagagcttctttacgagcgcattggggtgcttgctagcgcgtgcgctgtaggtcaattgcgacttggaagacatggtgccgcggcaatgaggatcatctgttagccattccatcaacaggaagaacgagagaaggcatgatccttttcgctggtattatccagatcaagttttagccgtataatctcagaacgaacccagtccatcgatgccatgtccttctagactaggatcctagagtctagggcccagcttagggagggcatgtgaatgcatcgatgactgggaacgaacaccggcccacgccaaagacgttacctaagataccttgatcattgtgagagtccagccaaaagtattccatgacttccatcgtatgccctctagagggctaatcgaggagtgtatttacattgtcggttggtttgggaactatagaagatggtcagttattccaatcaccaaaggtttatcgaagggaggaagacttgttcagtttcgtccgaggacttttggaattcaaatctgagatagagaattgtgtgggat

SEQ ID NO: 12 - DNA, 10235 bp, artificial sequence
  Plasmid pAUT657; figure 9.
gagaggaaaaggaagaagatggtggggttcagaaggaggggttgagttaaatagcatgggttgagtcaacgtgataagggcactataccgtatagatcagcggcacccgattctatccgttccttttgctcctctttagctttgaccggtgagccggacaagaaacaagtgaaatcatcctgacatcggcggacgatctcctagcttttacatttcgttaccaatgggatcccgtaatcaattgcccgtctgtcagatcgaggaatggtggatcatctgatccactatataacatagtactctgtccacggttagttaacatttatccaaaaacagaaaatctccacgactggaaccttttcaatagtgaaacctaagtattagcaaataatcgttacccccactcgcactccataatccttgatccagttctttttctttccgactttctcgactttctccgccctttcctgtaagctgtcaagagagaacgcggcgacagccacatcgttgttgcatttcactttgagacgcagcatatcgcactcgtctttcttcttcacaatggattcccattcagtttccaatcggatcctccaatgcatgtccgacacgcggccggatggatgcacgtctccaattagggtcgctacccgacccaagtactgacaccaccatgcctcctgcttagagacacaccggctcaattgccggtgaaattcttcatcggtgggtgcaaggtcccctgcccagcgctcacagagtatcaaggcaatcagggcacgttcggtgtgtgatagtgtatttaccgaaggcgcgccccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactaccatgcgctggaagttcaccctctccttcctcctcctcctctccggccgcgccctcgccttcccggcagggaatgccacggagctcgaaaagcgacagacaacccccaactcggagggctggcacgatggttattactattcctggtggagtgacggtggagcgcaggccacgtacaccaacctggaaggcggcacctacgagatcagctggggagatggcggtaacctcgtcggtggaaagggctggaaccccggcctgaacgcaagagccatccactttgagggtgtttaccagccaaacggcaacagctaccttgcggtctacggttggacccgcaacccgctggtcgagtattacatcgtcgagaactttggcacctatgatccttcctccggtgctaccgatctaggaactgtcgagtgcgacggtagcatctatcgactcggcaagaccactcgcgtcaacgcacctagcatcgacggcacccaaaccttcgaccaatactggtcggtccgccaggacaagcgcaccagcggtaccgtccagacgggctgccacttcgacgcctgggctcgcgctggtttgaatgtcaacggtgaccactactaccagatcgttgcaacggagggctacttcagcagcggctatgctcgcatcaccgttgctgacgtgggctaactcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcttaattaattggcggtgatattgatggcacgatagaagcagcacattctgtccctagacttagagattatcatgaagatattctcgatcaaatgctttttgcgctctttctcagcaaaagacatgctgatgcagcgaagctgctggaaagtattttcggtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatcccaatttagaagaagtgaaggtctgattctttccatccttccgccaacagtatcctccgagccgattcttccatggctggcggaccacaaatcaggaccatactctcatcttctggagccgcgtactcctttaggagctcttcggatatgcgtcctcggcggccagtccatgagtccggcgctttggatagggtgtgtattatattacaccttctgctgtcggttgccatgaagccgtcgagctcagcccggcaaaggatatcttcctcctgtctgtttccattgaggactgtacaagaggtgggatcttgccggtcctgaaccacggcgcgcaagacctggaagatcggtgtgataccggttcctccacaaatcatcttaaacgaccgaacatggcgttccttcccacttatgacaactcgtccatttccaaggtattcgaatctgcctgtcggacccttgcattccaccacggagcccaatggcagcctatccagggccatcgtcatcttgccgcctgccgaggtggctgttgcaaagtatactttaaccagcaagtccacggtccctttctggctggtttcagaaattggggtgtatgagcggatgatggcttcgttgttggatgatgtgtcgaggactttgatcataagatgctggccgactggtaaacccaatgtttgatcttcgtgttccaatttgaaactaaatattcgtgtatcccaggatatgtctttcctttctttcaatgttgcctttgtccaagaccgtgattggaggaacactgggcgaatttcatcggtggaggatgatgcatcatccttgagtgcttttaaaccttccgggtccatcgttccaatatggtactcaggcatcatcgcctttgccgtctcgctatctatggataggtgtcaatagatggtacaattgcagtgtgatatttttgggactcacgaatagcaaggaattcctcagagacatccagaccagcagaggagataatactctgcgctccgccagggtggccttcaagaaatgcttgaccatcatacacttctccattcacgatgaaccatggcttctcatcgcaggaattctccttgaattcttcaaaaccaatcactcggcttagcccgtctttcttcatattaatgtcttgcacgggctccggctccgtcggctcctctccttcgtgtctttctccccagttaccattcgtcaggtcacccccagcctttttgacgcgttccatccatcctgtaggcatactagggtgggtagggtgctcgaatctcaagttcccgttttccttcgtaattgtaacccggaaccacgggttgttcatcattccgagaacggaccagtacatatcgcgaggctgcacgcccaatgcttcgtccatggctcttacaaggatggcatcactgttctcaagctctgggatggtgatgcttagagaccaaaaacaccagcagaagcaagtttcgcgccagtacatatctactttgcctccaaaaagctcgccttcaaaatcacgatacttgtcttcggcatattcgatttccgccaatctccaagctataagtccgttagctttgataagcattctcacacatcgagcgagcgagggtgcgtacatttgcctttgtctagggatatttctaccctggtaaccctgcggcccccaccggcgtatgcatatcctctgacagtatatgacggccctgccgacaggagatttaagacctcattgttttggggatatgcaacggcggagttggtgtttaggtcataaatcgcataccgctcatcgtgccaccaatttcggttatttgatgccatctcaggcgagaccattgttctgggttagggagttagacaaatgatggaaatataaaataagtgccctttagacatacggtaagacgcggttgtcattgatatggtaccagttgtcgcttggtgcatcggtcaagatcagcctcttcagccacttaacacttcgtcctcctatttgaccgggcacgacggccctcagcggacgaccatgatctgggcgaagagactccccgttcattttatgtgcaagcatgatccccctgttggggtccagggcccagttcaatttaatagatgtgccgtagtgaccattgggctgcggcgaacttagcaattatcatcataagatagaggtacagcataccagcttatccgctccttccatacagacgtatttcgctttacgcaggggtttcgcactgcggagaatatccgccagcaatgggccagtgaagagggcagtcgatagtcccgccgatccccaggaaaaacctttcgttttacgtacaatgttttgctctttgcgtcgattgccagcacatacgagggtgataggcgctgttatttggtcgtactgctgcaacacttgtcggaagtttagtaccaaaggcttctctaccagtctatactttggttaacggatgtttggcagagaacctagcactatactaacccttcgatgctaatttcccagtgagggatatcttcatccttgatatgagggactgggccatgatttcgaacatagaagagctccggcgatgttaaaaaccctttcagagtgtgagaatgtaacggctcaaggggacaagcatgacagccggtgcaagcaacctgataaggataggagtggagcagttataactcataccttctttatacagatctcgatcgagctcgcgaaagcttactagtgcatgcgtttctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccgacaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagactagtgcatgccctagggtcgacttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactgccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatactcttcctttttcaatattattgaagcatttatcagggttattgtctcatgagcggatacatatttgaatgtataaactagtgcatgcaagcttattagtgataccccactctaagaaaatagaccaatctccagctgcaccttcagacactccggtacaaattctcgtctatgttggagattgttgtgactttgaaacatgacccttgaccctgattttgaatttgtccatatatcgaggcaggtgtcttattcgtacggagagggtatctgtcgtagacacatagtagtagtcatttcgagtgctgaatttataaatcgcatcatacttgcgacatactgccataaaaggagtacgtatccaccactacttattgcgcaccaacacgcttcaggtatgcatcccatccctccttctggtactgcttcgccgcctccacgggatcaggagcagcataaattccacggccagcaataataaagtcggcaccgcgtccaacagccgactcaggagtttggtactgctgtcccagcttgtcacccttcgaggagaggttgacacctgtcgtgaagacgacaaaatcttcctcctccgaaggcgagctaacttcagactgaacctcgccaaggtgacgtgtcgagacgaatcccatcacaaacttcttatacttccgagcatagtcaacagaagaagtagtatattgaccggtagccaaagatcccttggaggtcatctccgcaaggatcaaaaggcccctctcggagccgtaggggaagtcctcggccgaagcagtctgggccagagcctcgacgataccctcaccgggcagaatactgcagttgatgatgtgggcccactcagagatacgcagagtgccgccatggtactgcttttggactgtgtttccgatatcgatgaacttgcgatcttcgaagatgaggaaattgtgcttctctgcaagggccttcagaccggtgatggtttcttcgctgaaatcggagaggatatcgatgtgagttttgatcacggcaatgtacggaccgagtcctgttatataatccaccattaaccattactagatcacatgtaagtggcatccccggtgcgcatacggtcagccaaatccagcagctctttggtggttgtcacgtcggcggaaacggtgacattggttttcttggcctcggcaacctcgaagagcttctttacgagcgcattggggtgcttgctagcgcgtgcgctgtaggtcaattgcgacttggaagacatggtgccgcggcaatgaggatcatctgttagccattccatcaacaggaagaacgagagaaggcatgatccttttcgctggtattatccagatcaagttttagccgtataatctcagaacgaacccagtccatcgatgccatgtccttctagactaggatcctagagtctagggcccagcttagggagggcatgtgaatgcatcgatgactgggaacgaacaccggcccacgccaaagacgttacctaagataccttgatcattgtgagagtccagccaaaagtattccatgacttccatcgtatgccctctagagggctaatcgaggagtgtatttacattgtcggttggtttgggaactatagaagatggtcagttattccaatcaccaaaggtttatcgaagggaggaagacttgttcagtttcgtccgaggacttttggaattcaaatctgagatagagaattgtgtgggat

(19) United States
(12) Patent Application Publication
(10) Pub. No.: US 2022/0267783 A1
(43) Pub. Date: Aug. 25, 2022

(54) FILAMENTOUS FUNGAL EXPRESSION SYSTEM

(71) Applicant: Novozymes A/S, Bagsvaerd (DK)

(72) Inventor: Dominique Aubert Skovlund, Vaerloese (FR)

(73) Assignee: Novozymes A/S, Bagsvaerd (DK)

(21) Appl. No.: 17/630,107

(22) PCT Filed: Jun. 25, 2020
(86) PCT No.: PCT/EP2020/067823

§ 371 Date: Jan. 25, 2022

(30) Foreign Application Priority Data
Jul. 25, 2019 (EP) 19188253.9

(51) Int. Cl. C12N 15/80; C12N 9/20; C12N 9/24; C12N 9/88; C12N 9/90; C12N 9/00

(52) U.S. Cl. CPC C12N 15/80; C12N 9/20; C12N 9/248; C12N 9/88; C12N 9/90; C12N 9/93; C12N 2330/51

(57) ABSTRACT

The present invention provides recombinant filamentous fungal host cells producing one or more secreted polypeptide of interest, said cells comprising in their genome at least one nucleic acid construct comprising a first polynucleotide encoding a signal peptide operably linked in translational fusion to a second polynucleotide encoding the polypeptide of interest, wherein the first polynucleotide is heterologous to the second polynucleotide, wherein the first polynucleotide is a polynucleotide having at least 70% sequence identity with SEQ ID NO:1 or a polynucleotide encoding a signal peptide having at least 70% sequence identity with SEQ ID NO:2, as well as methods of producing one or more secreted polypeptide of interest.

### REFERENCE TO SEQUENCE LISTING

This application contains a Sequence Listing in computer readable form. The computer readable form is incorporated herein by reference.

### FIELD OF THE INVENTION

The present invention relates to filamentous fungal expression systems, in particular to the expression of one or more secreted polypeptides of interest in translational fusion with a heterologous signal peptide of the invention.

### BACKGROUND OF THE INVENTION

Product development in industrial biotechnology includes a continuous challenge to increase enzyme yields at large scale to reduce costs. Two major approaches have been used for this purpose in the last decades. The first one is based on classical mutagenesis and screening. Here, the specific genetic modification is not pre-defined and the main requirement is a screening assay that is sensitive to detect increments in yield. High throughput screening enables large numbers of mutants to be screened in search for the desired phenotype, i.e., higher enzyme yields. The second approach includes numerous strategies ranging from the use of stronger promoters and multicopy strains to ensure high expression of the gene of interest to the use of codon optimized gene sequences to aid translation. However, high level production of a protein may trigger several bottlenecks in the cellular machinery for secretion of the enzyme of interest into the medium.

Signal peptides (SPs) are short amino acid sequences present in the amino terminus of many newly synthesized proteins that target proteins into, or across, membranes e.g., the endoplasmic reticulum (ER). Bioinformatic tools can predict SPs from amino acid sequences, but most cannot distinguish between various types of SPs (Armenteros et al., 2019). A large degree of redundancy in the amino acid sequence of SPs makes it difficult to predict the efficiency of any given SP for production of enzymes at industrial scale. For secreted enzymes containing an SP, translation is followed by cleavage of the SP by a signal peptidase and translocation of the maturing protein into the ER (Voss et al., 2013; Aviram and Schuldiner 2017). To secrete a protein through the ER, the signal recognition particle (SRP) recognizes the SP in a highly conserved manner. The SRP associates with the ribosome and through a hydrophobic cleft recognizes secretory proteins with hydrophobic motifs as they are being translated and binds to the SRP receptor (SR) present in the ER membrane in eukaryotes (Aviram and Schuldiner 2017). The amino acid sequence of the SP may influence secretion efficiency and thereby the yield of the enzyme manufacturing process.

SPs include three functional domains 1) the n-region at the N-terminal region of the SP that normally displays a net positive charge as a result of the presence of one or two basic residues (K, R), 2) the hydrophobic (h) region whose length and level of hydrophobicity may determine the affinity of the SP towards the protein secretion pathway and the polar region (c-region) where the cleavage site for signal peptidase (e.g., AXA at position −3 to −1, cleavage after the second A) is located (Low et al., 2013, FIG. 1). In addition, SPs also display a pro-region that at least in bacteria may extend from position +1 to +6. The pro-region requires a net negative charge.

Many algorithms to predict SPs and their cleavage sites from amino acid sequences have been developed based on artificial neural networks (NN) or hidden Markov models (HMM, Armenteros et al., 2019). SignalP is one of the first developed and more advanced methods for in silico identification of SP candidates. A recent update, SignalP5, can predict proteome-wide SPs across all organisms, and classify them into different SP types (Armenteros et al., 2019). Still, selection of SP is known to be an important step for manufacturing of recombinant proteins.

Screening of homologous SPs in bacterial hosts indicated that the optimal SP for one protein may be rather inefficient for another protein. No correlation with the n-region net charge, hydrophobicity level or length could be identified for the high secretion performance SPs (Low et al., 2013).

To add more complexity, a considerable number of secreted fungal proteins are synthesized as pro-proteins undergoing proteolytic processing within the secretion pathway (Punt et al., 2003).

### SUMMARY OF THE INVENTION

It is the object of the present invention to provide improved methods for producing a secreted polypeptide in a filamentous fungal host cell. We have modified and used a signal peptide denoted SP17 having the amino acid sequence shown in SEQ ID NO:2 (with or without the final N-terminal alanine) which is encoded by SEQ ID NO:1 (with or without the final “gcc” codon), originally identified in Aspergillus oryzae, to construct strains that produce and secrete significantly increased amounts of a heterologous xylanase compared to widely used benchmark SPs in comparable A. oryzae strains. The increase in xylanase production was consistently observed at different scales from microtiter plates to lab scale tank fermentation.

In a first aspect, the invention relates to recombinant filamentous fungal host cells producing one or more secreted polypeptide of interest, said cells comprising in their genome at least one nucleic acid construct comprising a first polynucleotide encoding a signal peptide operably linked in translational fusion to a second polynucleotide encoding the polypeptide of interest, wherein the first polynucleotide is heterologous to the second polynucleotide, wherein the first polynucleotide is selected from the group consisting of:
a) a polynucleotide having at least 70% sequence identity with SEQ ID NO:1; preferably at least 75% sequence identity with SEQ ID NO:1; or preferably at least 80% sequence identity with SEQ ID NO:1; preferably at least 85% sequence identity with SEQ ID NO:1; or preferably at least 90% sequence identity with SEQ ID NO:1; preferably at least 95% sequence identity with SEQ ID NO:1; or at least 97% sequence identity with SEQ ID NO:1; or most preferably at least 99% sequence identity with SEQ ID NO:1; and
b) a polynucleotide encoding a signal peptide having at least 70% sequence identity with SEQ ID NO:2; preferably at least 75% sequence identity with SEQ ID NO:2; or preferably at least 80% sequence identity with SEQ ID NO:2; preferably at least 85% sequence identity with SEQ ID NO:2; or preferably at least 90% sequence identity with SEQ ID NO:2;

preferably at least 95% sequence identity with SEQ ID NO:2; or at least 97% sequence identity with SEQ ID NO:2; or most preferably at least 99% sequence identity with SEQ ID NO:2.

In a second aspect, the invention relates to methods of producing one or more secreted polypeptide of interest, said method comprising the steps of:
a) cultivating a recombinant filamentous fungal host cell as defined in the first aspect under conditions conducive to the production of the polypeptide of interest and, optionally,
b) recovering the polypeptide of interest.

### BRIEF DESCRIPTION OF DRAWINGS

FIG. 1 shows the structure and favored amino acid positions in eukaryotic SPs. The predicted cleavage site is depicted with a red vertical line. Position of the n-, h and c-regions are depicted as labelled double arrows above. Sequence logo in one letter amino acid code is taken from the background information at the SignalP server (http://www.cbs.dtu.dk/services/SignalP-3.0/background/dataset.php).

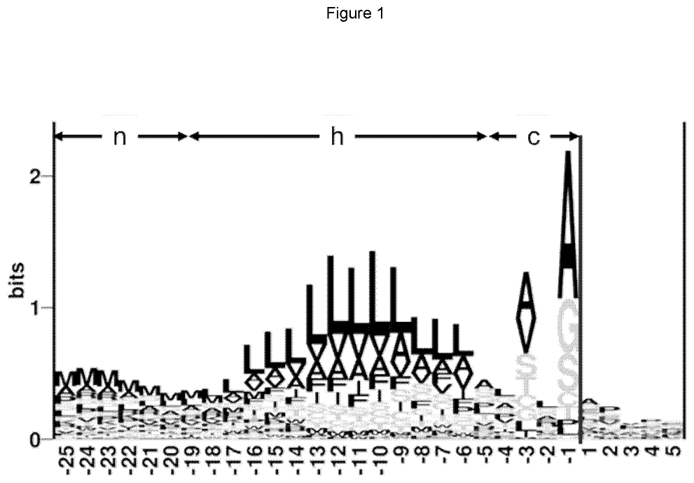

FIG. 2 shows the generic cloning strategy for SP plasmid construction used herein for the cloning of the SP17 in this work. Digestion of the vector with NaeI and XhoI enables cloning of the Gene of Interest (GOI) for example the CDS of the xInTL gene consisting of a PCR fragment cut with XhoI and left blunt at the 5′end). This allows an in-frame fusion of the SP and xylanase gene, xInTL. An Alanine codon was added, if not present at the C-terminus of the SP sequence, as shown in Example 1.

FIG. 3 shows Xylanase activity (in U/ml) measured in the supernatant of the MTP fermentation of strains transformed with plasmids with the different SP constructions in Example 1. Eight strains (1-8) were isolated for each SP construct and fermented in 96 wells MTP. Data for each SP is arranged from lowest to highest producing strain. Plasmid pAUT751 contains the xylanase gene with its native SP (wt SP), plasmids pAUT654, and pAUT657 contain the pro and mature xInTL region with SP17 and SP20, respectively. A dotted line at about 15 U/ml is indicated at the level of activity consistently reached by the control strain JaL339 in these fermentation conditions.

FIG. 4 shows the correlation between xInTL copy number and xylanase activity at the end of fermentation (167 h) for strain AUT812, AUT805, AUT806, AUT813 and AUT810 having an increasing number of copies (9-36) as indicated; Example 2. Xylanase activity shown in grey boxes (values at left axis). Copy no. shown in black boxes (values at right axis).

FIG. 5 shows the xylanase activity comparison between the control strain JaL339 and SP17 (AUT805, AUT806) as well as SP20 (AUT807, AUT808) strains containing different copy numbers of the xInTL gene, at the end of fermentation (167 h) in Example 2. Xylanase activity shown in grey boxes (values at left axis). Copy no. shown in black boxes (values at right axis).

FIG. 6 shows a schematic plasmid map of plasmid pJaL537 (SEQ ID NO:9).

FIG. 7 shows a schematic plasmid map of plasmid pAUT751 (SEQ ID NO:10).

FIG. 8 shows a schematic plasmid map of plasmid pAUT654 (SEQ ID NO:11).

FIG. 9 shows a schematic plasmid map of plasmid pAUT657 (SEQ ID NO:12).

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic having one or more (e.g., several) amino acids absent from the amino and/or carboxyl terminus of a mature polypeptide or domain; wherein the fragment retains its enzyme activity.

Host cell: The term “host cell” means any filamentous fungal cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase. However, as we have shown herein, the selection of a specific signal peptide may provide surprising improvements in the yield or productivity of heterologous secreted polypeptides of interest. The SP17 signal peptide of the instant invention is one example.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The filamentous fungal host cell of the invention may be any filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucormiehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma Trichoderma longibrachiatum, Trichoderma reesei, or Trichoderma viride cell.

Preferably, the filamentous fungal host cell is an Aspergillus oryzae cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

In a first aspect, the invention relates to recombinant filamentous fungal host cells producing one or more secreted polypeptide of interest, said cells comprising in their genome at least one nucleic acid construct comprising a first polynucleotide encoding a signal peptide operably linked in translational fusion to a second polynucleotide encoding the polypeptide of interest, wherein the first polynucleotide is heterologous to the second polynucleotide, wherein the first polynucleotide is selected from the group consisting of:
a) a polynucleotide having at least 70% sequence identity with SEQ ID NO:1; preferably at least 75% sequence identity with SEQ ID NO:1; or preferably at least 80% sequence identity with SEQ ID NO:1; preferably at least 85% sequence identity with SEQ ID NO:1; or preferably at least 90% sequence identity with SEQ ID NO:1; preferably at least 95% sequence identity with SEQ ID NO:1; or at least 97% sequence identity with SEQ ID NO:1; or most preferably at least 99% sequence identity with SEQ ID NO:1; and
b) a polynucleotide encoding a signal peptide having at least 70% sequence identity with SEQ ID NO:2; preferably at least 75% sequence identity with SEQ ID NO:2; or preferably at least 80% sequence identity with SEQ ID NO:2; preferably at least 85% sequence identity with SEQ ID NO:2; or preferably at least 90% sequence identity with SEQ ID NO:2; preferably at least 95% sequence identity with SEQ ID NO:2; or at least 97% sequence identity with SEQ ID NO:2; or most preferably at least 99% sequence identity with SEQ ID NO:2.

It is expected that the invention will be just as effective when employing a signal peptide that is highly similar to the SP17 signal peptide disclosed in SEQ ID NO:2 and encoded by SEQ ID NO:1. One or more non-essential amino acids may, for example, be altered. Non-essential amino acids in a signal peptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for signal peptide activity to identify amino acid residues that are critical to the activity of the molecule and residues that are non-essential. See also, Hilton et al., 1996, J. Biol. Chem. 271: 4699-4708. The identity of essential and non-essential amino acids can also be inferred from an alignment with one or more related signal peptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In a preferred embodiment, the first polynucleotide encodes a signal peptide comprising or consisting of the amino acid sequence of SEQ ID NO:2.

In another preferred embodiment, the first polynucleotide encodes a signal peptide consisting of the amino acid sequence of SEQ ID NO:2 with or without its C-terminal alanine, or a peptide fragment thereof that retains the ability to direct the polypeptide into or across a cell membrane. Correspondingly, it is preferred that the first polynucleotide comprises or consists of SEQ ID NO:1 with or without its 5′ gcc codon, or a subsequence thereof which encodes a signal peptide that retains the ability to direct the polypeptide into or across a cell membrane.

The invention is expected to work for all secreted polypeptides irrespective of whether or not they are native to the host cell. Accordingly, it is preferred that the second polynucleotide encodes a polypeptide that is native or heterologous to the filamentous fungal host cell.

Preferably, the second polynucleotide encodes an enzyme; more preferably the second nucleotide encodes an oxidoreductase, transferase, hydrolase, lyase, isomerase, or ligase; and most preferably the second nucleotide encodes an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

Preferably, the second polynucleotide encodes a xylanase and comprises or consists of a nucleotide sequence having at least 70% sequence identity with SEQ ID NO:7; preferably at least 75% sequence identity with SEQ ID NO:7; preferably at least 80% sequence identity with SEQ ID NO:7; preferably at least 85% sequence identity with SEQ ID NO:7; preferably at least 90% sequence identity with SEQ ID NO:7; preferably at least 95% sequence identity with SEQ ID NO:7; preferably at least 97% sequence identity with SEQ ID NO:7; or preferably at least 99% sequence identity with SEQ ID NO:7. Even more preferably, the second polynucleotide encodes a xylanase and comprises, consists essentially of, or consists of SEQ ID NO:7.

Preferably, the second polynucleotide encodes a xylanase having at least 70% sequence identity with SEQ ID NO:8; preferably at least 75% sequence identity with SEQ ID NO:8; preferably at least 80% sequence identity with SEQ ID NO:8; preferably at least 85% sequence identity with SEQ ID NO:8; preferably at least 90% sequence identity with SEQ ID NO:8; preferably at least 95% sequence identity with SEQ ID NO:8; preferably at least 97% sequence identity with SEQ ID NO:8; or preferably at least 99% sequence identity with SEQ ID NO:8. Even more preferably, the second polynucleotide encodes a xylanase comprising, consisting essentially of, or consisting of SEQ ID NO: 8.

### Methods of Production

The second aspect of the invention relates to methods of producing one or more secreted polypeptide of interest, said method comprising the steps of:
a) cultivating a recombinant filamentous fungal host cell as defined in the first aspect under conditions conducive to the production of the polypeptide of interest and, optionally,
b) recovering the polypeptide of interest. The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### EXAMPLES

### Materials and Methods

General methods of PCR, cloning, ligation, nucleotides etc. are well-known to a person skilled in the art and may for example be found in ‘Molecular Cloning: A Laboratory Manual’, Sambrook et al. (1988), Cold Spring Harbor Lab., Cold Spring Harbor, N.Y.; Ausubel, F. M. et al. (eds.); ‘Current Protocols in Molecular Biology’, John Wiley and Sons (1995); Harwood , C. R., and Cutting, S. M. (eds.); ‘DNA Cloning: A Practical Approach, Volumes I and II’, D. N. Glover ed. (1985); Oligonucleotide Synthesis', M. J. Gait ed. (1984); ‘Nucleic Acid Hybridization’, B. D. Hames & S. J. Higgins eds (1985); ‘A Practical Guide To Molecular Cloning’, B. Perbal (1984).

### Plasmid Construction and Plasmid Description

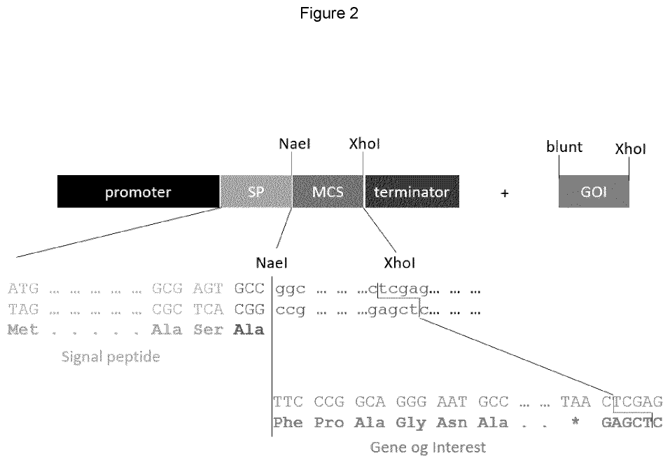

The cloning strategy is designed to enable cloning of different genes of interest (GOI) and SP sequences and is based on restriction ligation of DNA fragments (FIG. 2).

Cloning in the Nael site (blunt-end) will generate an extra codon (GCC) that codes for Alanine (Ala or A). This extra codon is added to the SP sequence for SP17 (Table 2). This cloning strategy is a generic process based on the same restriction enzyme combination as well as a unique fragment carrying the xInTL gene—or any other gene of interest that can be cloned in the SP plasmid cut by NaeI and XhoI. Although this may affect SP cleavage or result in a mature protein with an added A at the N-terminus, we expected that xylanase activity was the best measure for secretion and enzyme yield efficiency regardless the modified SP sequences.

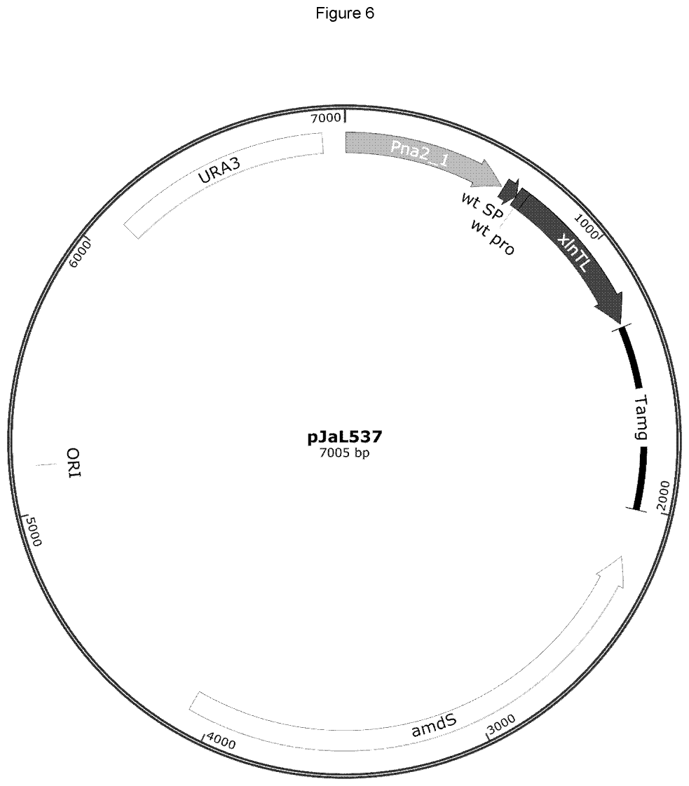

The original plasmid used for construction of a XInTL production strain is pJaL537 (Table 1, FIG. 6). In this plasmid, expression is controlled by the Pna2 promoter (herein referred to as Pna2_1) that is derived from the A. niger neutral amylase amyB gene. Pna2-1 is induced by maltose and repressed by e.g., glycerol. In pJaL537, secretion of XInTL is driven by its native SP (wtSP).

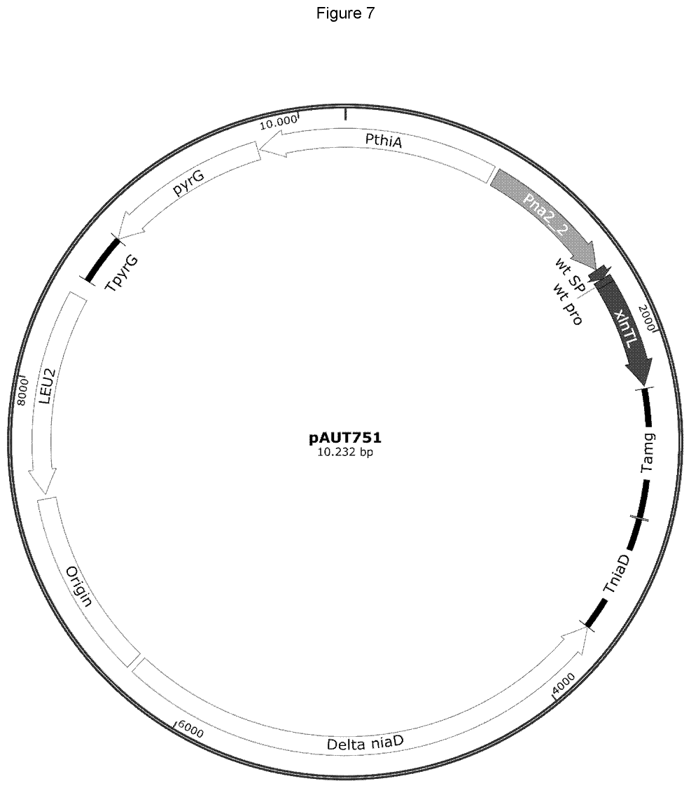

Plasmids (pAUT751, pAUT654, and pAUT657, Table 1; FIGS. 7, 8 and 9, respectively) contain a SP (wtSP, SP17 and SP20, respectively) and a slightly modified and stronger Pna2 promoter (Pna2_2). Introduction of the plasmids in A. oryzae is based on a transformation system where the expression cassette is introduced in multiple copies to complement a truncated niaD gene encoding nitrate reductase present in the genome of the recipient strain AT1100 (Olsen 2013). In this system, a pyrG gene is introduced in the transforming plasmid. High copy number strains can be generated by growth in medium with nitrate as sole nitrogen source (requires a functional niaD gene, following homologous recombination of the plasmid at the niaD locus). Using nitrate selection alone, multiple copies of the plasmid (typically between 3-8) containing the xInTL expression cassette can be obtained. This is relevant to test for possible detrimental effects of multiple copies in an initial screening. Higher copy numbers can be obtained by combining nitrate selection with the addition of thiamine to the growth medium. Expression of the pyrG gene present in the plasmid is regulated by the P_{thiA}, the promoter of the thiamine biosynthetic gene thiA, Olsen 2013).

Expression of pyrG is greatly reduced in the presence of thiamine. No addition of uridine is used to the transformation plates, to select for expression of pyrG to sustain growth in the presence of thiamine. In this way, strains containing high number of the plasmid (10-50) can be obtained.

TABLE 1
Genetic elements used for expression and secretion of
the T. lanuginosus XInTL xylanase in A. oryzae.

Genetic elements

| Plasmid | Promoter | SP |
|---|---|---|
| pJaL537 | Pna2_1 | wtSP |
| pAUT751 | Pna2_2 | wtSP |
| pAUT654 | Pna2_2 | SP17 |
| pAUT657 | Pna2_2 | SP20 |

### Aspergillus Transformation

Transformation of Aspergillus oryzae was done as described in U.S. Pat. No. 9,487,767. Transformants that had repaired the target niaD-gene and contained the pyrG gene were selected for its ability to grow on minimal plates containing nitrate as nitrogen source (Cove, 1966). To obtain integration of higher copy numbers of the expression cassette, thiamine was added to the medium, reducing expression of pyrG. After 5-7 days of growth at 30° C., stable transformants appeared as vigorously growing and sporulating colonies. Transformants were purified through conidiation. Strains obtained by this method may contain different copy numbers of the expression cassette integrated head-to-tail at the niaD locus. Thus, screening of individual transformants may include strains with different copy numbers.

### Strain Cultivation

The transformed cells are cultivated in a nutrient medium suitable for production of the recombinant protein using methods well known in the art. For example, the cells may be cultivated by shake flask cultivation (in which 10 mL YPD medium (2 g/L yeast extract, 2 g/L peptone and 2% glucose) were inoculated with spores from a transformant and incubated at 30° C. for 4 days), and small-scale (Microtiter Plate (MTP) cultivation) or lab-scale fermentation (including e.g., batch or fed-batch fermentation) in laboratory or industrial fermentor performed in a suitable medium and under conditions allowing the recombinant protein to be expressed and recovered. The cultivation takes place in a suitable nutrient medium comprising carbon and nitrogen sources and inorganic salts, using procedures known in the art. Suitable media are available from commercial suppliers or may be prepared according to the manufacturer's recommendation. The recombinant protein is secreted into the nutrient medium and can be recovered directly in the culture supernatant.

Copy Number Determination by ddPCR

Copy number determination was performed by ddPCR using BioRad QX200™ Droplet Generator and QX200™ Droplet Reader using Biorad QuantaSoft™ version 1.7.4.0917, according to the manufacturer, using a probe derived from A. oryzae oliC as single copy gene reference.

### Enzyme Assay

Xylanases hydrolyze wheat arabinoxylan (Megazyme) to release reducing sugars. The reaction is stopped by an alkaline solution containing PAHBAH (para-hydroxybenzoic acid hydrazide) and Bismuth which complexes with reducing sugar, producing colour that is detected at 405 nm. The colour is proportional to xylanase activity and is measured relative to an enzyme standard. Culture supernatants were used to measure xylanase activity. Samples and standards (20 μl) were incubated with 110 μl of a 0.5% solution of arabinoxylan. The reaction was performed at pH 6.0 at room temperature for 30 min. Reactions were stopped by addition of 100 μl of the alkaline solution and further incubated for 15 min. The samples are measured in a plate reader as an endpoint reading (Molecular Devices) and absorbance measured simultaneously. Preparation of samples for activity measurement was performed on a Hamilton Star plus liquid handler. Samples were diluted and assayed in 96 well microtitre plates to fit within the standard curve.

### Strains

The A. oryzae host strain AT1100 is derived from BECh2 which is described elsewhere (Christiansen et al. 2000). JaL339 is also a strain derived from BECh2 producing T. lanuginosus xylanase strains constructed using a similar expression cassette using ectopic integration (JaL339). All other strains described here were constructed using a homologous recombination, multicopy integration method described elsewhere (Table 1, Olsen 2013).

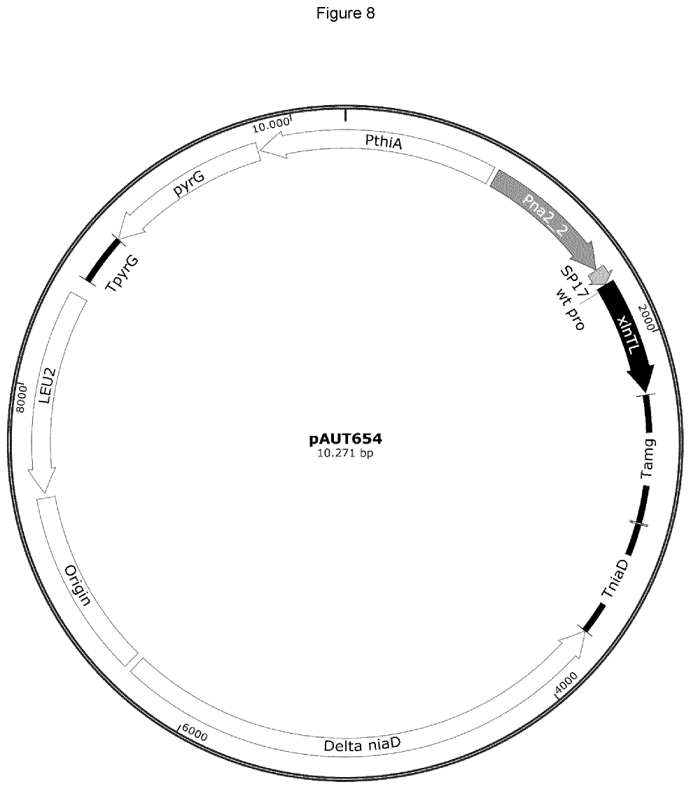

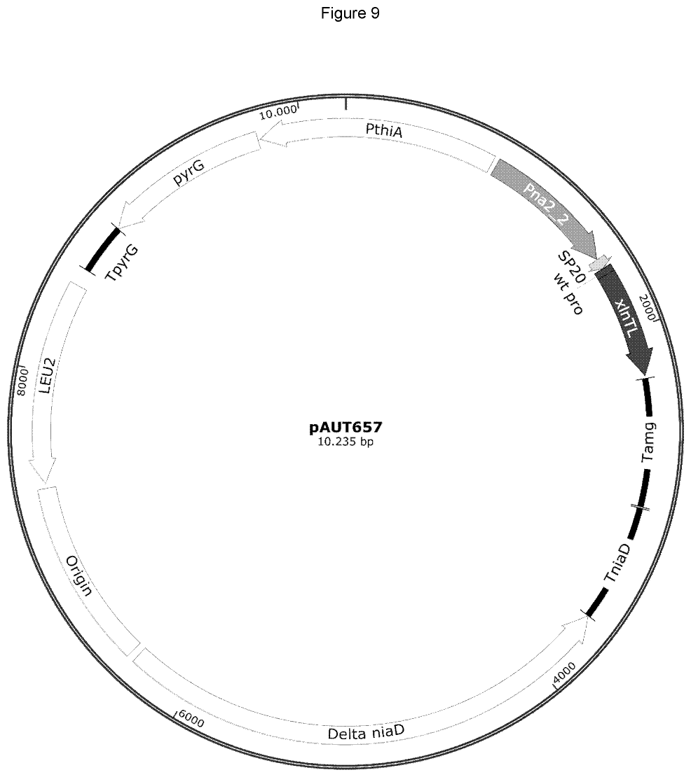

A control construction—to compare the contribution of the promoter and the selection system to the original strain JaL339—contained the wt xInTL coupled to its native wild type (wtSP in pAUT751, FIG. 7). As mentioned above, in pAUT654 (FIG. 8) and pUT657 (FIG. 9), a slightly modified promoter (Pna2_2) is used to drive expression of xInTL with either SP17 or SP20, respectively.

Strains with different copy numbers (xInTL copy #; Table 2) that were selected for the final yield comparison in lab tanks are described together with the plasmid and the signal peptide used for strain construction and the copy number (Table 2). Other strains are described in the Example sections below.

TABLE 2
Strains and plasmids used in this work.

| | | | | xInTL |
|---|---|---|---|---|
| Strain Name | host | plasmid | Signal peptide | copy # |
| JaL339 | BECh2 | pJAL537 | Wild type SP xInTL; SEQ ID NOs: 5/6 | 44 |
| AUT805 | AT1100 | pAUT654 | SP17; SEQ ID NOs: 1/2 with Ala | 8 |
| AUT806 | AT1100 | pAUT654 | SP17; SEQ ID NOs: 1/2 with Ala | 27 |
| AUT807 | AT1100 | pAUT657 | SP20; SEQ ID NOs: 3/4 | 11 |
| AUT808 | AT1100 | pAUT657 | SP20; SEQ ID NOs: 3/4 | 22 |

### Xylanase Gene

The mature fungal Thermomyces lanuginosus xylanase enzyme (SEQ ID NO:8) used in this study is encoded by the xInTL gene without its native signal- and propeptides (SEQ ID NO:7). Its native signal- and propeptide sequence are shown in SEQ ID NO:6 encoded by SEQ ID NO:5.

### Example 1. Construction of Strains for Production of Xylanase in A. Oryzae using Selected SP sequences: Initial Benchmarking to the Xylanase Wild Type SP

Signal peptides (SPs) are found in many nascent polypeptides in virtually all organisms and are necessary for secretion of the protein to the target location. SPs are found in secreted and transmembrane (TM) proteins, as well as in proteins inside organelles in eukaryotic cells. The general secretory pathway (Sec) directs protein translocation across the plasma membrane in prokaryotes and the endoplasmic reticulum membrane in eukaryotes (Armenteros et al., 2019).

In order to identify the most suitable SP for production of the XInTL xylanase in A. oryzae, standard SPs used in industrial enzyme production in fungi that include the Coprinus cinereus cutinase SP (Matsui et al. 2014), the T. lanuginosus lipase SP (Yaver et al., 2007) and a SP derived from plectasin (an antimicrobial defensin produced by the ascomycete Pseudoplectania nigrella (Mygind et al 2005) may be used. Other SP sequences have also been used although their relevance, sequence modification and functionality remain unclear (Toida et al., 2000). In this paper, they state that the signal sequence of the tgIA gene encoding a triacylglycerol lipase is concluded to span from the initial Methionine to Arginine at position 30 and that the same cleavage sites after an R were found in other Aspergillus secreted enzymes. Another potential candidate for XInTL production is the SP described by Yano et al. (2008), derived from the Icc1 laccase gene from Lentinula edodes. Using this SP, secretion of non-secreted laccases in an active form was obtained in A. oryzae. Therefore, this SP (herein referred to as SP20) is a good candidate for benchmarking SP efficiency in A. oryzae. During an initial screening of some of the above mentioned published and a set of homologous SP (Skovlund et al., manuscript in preparation), we identified a modified version of the tgIA SP (herein referred to as SP17), that contains an extra Alanine added to the C-terminus of the SP and set out to compare yields to the wtSP (derived from the XInTL native (wt) sequence) and to another candidate identified in the initial screening (SP20, Table 3).

Secreted fungal proteins can be synthesized as pro-proteins that undergoing proteolytic processing of the pro-sequence during secretion (Punt et al., 2003). The length, position and composition of these pro-sequences is not completely understood, with the exception of the presence of a dibasic motif (e.g., KR) at the site of cleavage by the furin-type protease KexB (Punt et al., 2003).

TABLE 3
Sequence of the SPs used for xylanase
production A. oryzae in this work.

| | Sequence description | DNA/Amino |
|---|---|---|
| SP | (donor organism) | acid sequence |
| SP17 | TgIA Triacylglycerol lipase (A. oryzae)* | SEQ ID NOs: 1/2*: |
| | | 1/2* |
| SP20 | Lcc1 Laccase (L. edodes) | SEQ ID NOs: 3/4 |
| wtSP | XInTL Xylanase (T. lanuginosus) | SEQ ID NOs: 5/6 |

Note:
*An additional Alanine was added to the C-terminus of SP17.

Thus, the T. lanuginosus xInTL gene encoding a xylanase was cloned successfully in plasmids with the different SPs upstream of the xInTL gene as well as the xInTL gene with its wild type SP were transformed into strain AT1100 with selection on plates containing nitrate as sole nitrogen source without thiamine addition. Between 1-8 transformant were selected per plasmid giving a distribution of strains with different copy number (between 3-8) of the expression cassette for each SP (Olsen 2013). Transformants were selected and grown in 96 well MTP fermentation experiments to compare the overall yield to JaL339, the original control strain for production of xylanase benchmark to include also SP20.

Spores were harvested, and appropriate dilutions were inoculated in fermentation medium. The samples were incubated at 30° C. for 24 hours. Maltose, a known inducer of the Pna2 promoter in A. oryzae (Olsen 2013) was added to the fermentation and the plates are incubated for a further 5-day period. The strains picked and inoculated in MTP fermentation were evaluated for xylanase activity. Strains (1-8 individual transformants) for each SP were evaluated since they may contain different copy numbers of the expression cassette (Olsen 2013).

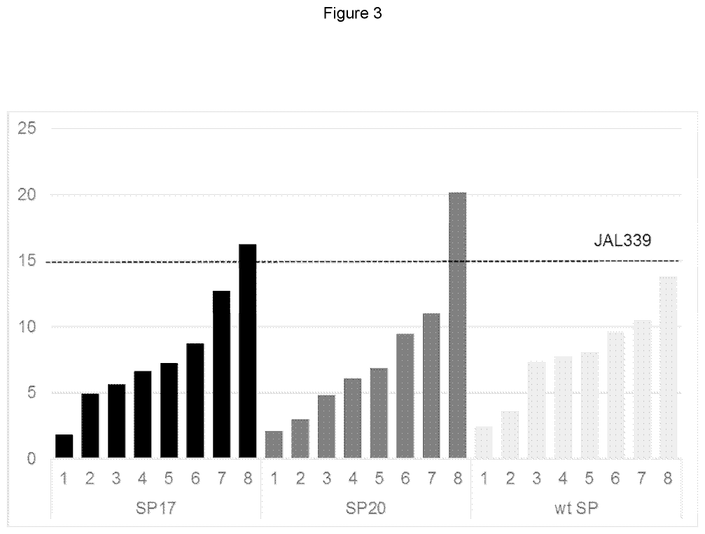

We observed that some of the strains produced a relatively good xylanase level. The strains obtained with control plasmid pAUT751 (containing the wt xylanase with its native SP) produced 2-14 xylanase U/ml. The maximum yield obtained is probably the result of a high copy number and is comparable to the yields normally obtained with control strain JAL339 (indicated as a dotted line, approx. 15 U/ml; FIG. 3),

Differences in xylanase activity between strains (in the range 2-20 U/ml, FIG. 3) may be due to the different copy number of the expression cassette. Therefore, measurement of the copy number of the xylanase expression cassette in the selected strains was analyzed by ddPCR. The copy number of the xylanase gene integrated in the strains obtained with the different SPs was quite low (3-8 copies) compared to the control strain JaL339 that contains 44 copies.

Overall, xylanase yields were obtained using SP17 and SP20 that represented an improvement compared to wtSP and to the yield of the original XInTL strain JaL339 (FIG. 3).

### Example 2. Use of SP17 for Construction of an Optimized Production Strain

To boost the number of copies of the xInTL expression cassette that can be integrated in the genome the SP plasmids (Example 1) were transformed in a new round of strain construction using strain AT1100 and thiamine selection. As mentioned above, addition of thiamine to the medium represses the promoter that drives expression of the selective marker pyrG leading to an increase in copy number of the integrated plasmid (Olsen 2013).

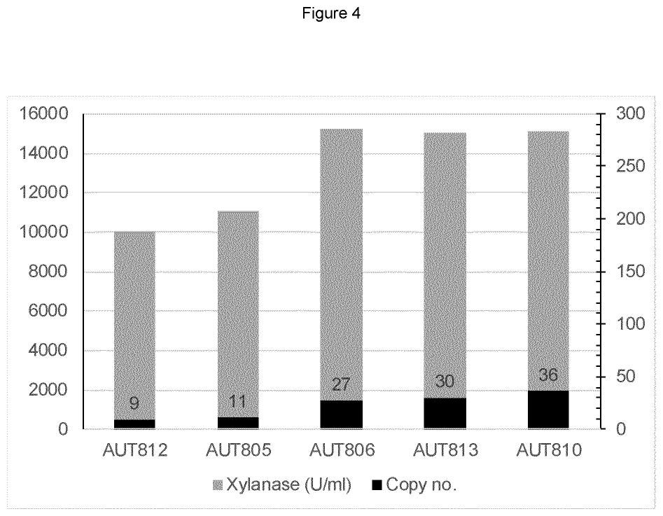

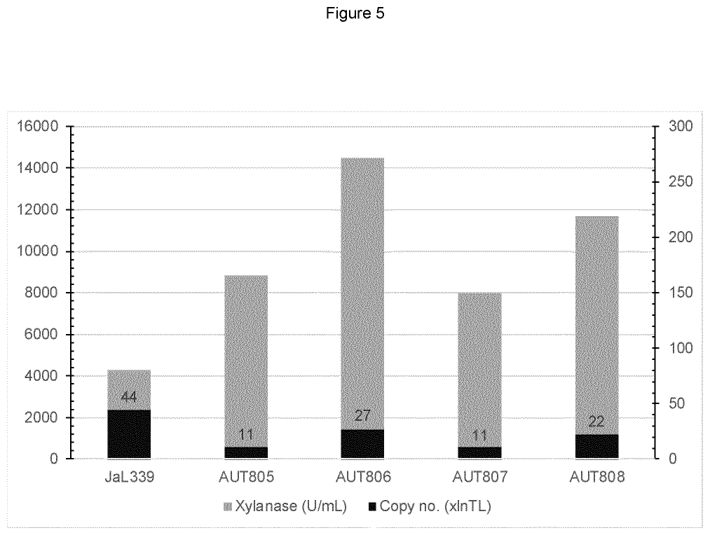

To correlate xylanase activity and copy number, strains transformed with pAUT654 (SP17) and selected on plates with thiamine were selected. The copy number of the xInTL gene was determined. Five strains containing 9-36 copies were tested for xylanase activity at the end of lab fermentation (167 h, FIG. 4). Significant yield increase was observed in the range 9-27 copies (strains AUT812, AUT805 and AUT806). Higher copy number (strains AUT813 and AUT810) did not lead to higher xylanase activity, indicating that strain AUT806 was a candidate for higher yield of xylanase. AUT805 and AUT806 were selected for a new round of lab fermentations (Table 4, FIG. 5).

Transformants obtained with plasmid pAUT657 (SP20) and thiamine selection were also analyzed for copy number to identify strains for comparison with SP17 strains AUT805 and AUT806 in lab tanks. Two strains for each of SP17 and SP20-xylanase together with the original xylanase strain JaL339 were chosen to be upscaled to lab scale fermentation (Table 4).

TABLE 4
Xylanase strains, SP and copy number of the
xInTL gene tested in lab fermentations.

| Strain Name | SP | Copy number 30 |
|---|---|---|
| JaL339 | wtSP | 44 |
| AUT805 | SP17 | 11 |
| AUT806 | SP17 | 27 |
| AUT807 | SP20 | 11 |
| AUT808 | SP20 | 22 |

The increase in copy number typically leads to an increase in product formation but it also reaches a maximum when transcription is not limiting, and other cellular processes become a bottleneck (Gressler et al., 2015).

The increase in xylanase activity for SP20 from 11 to 22 copies means that, in this range, more xInTL copies lead to more xylanase (FIG. 6). The same applies for SP17. Strain AUT805 having 11 copies of xInTL with SP17 yielded ca. 10% more than strain AUT807 with the same copy number and SP20. These results provide evidence that SP17 is superior to SP20 (and wtSP) for production of the XInTL xylanase in A. oryzae.

At high copy number, the increase in xylanase activity is maintained demonstrating that the use of SP17 improves xylanase yields and that strain AUT806 is an optimized strain yielding more than a 3-fold increase compared to JaL339.

### REFERENCES

Armenteros J J A et al. (2019) SignalP 5.0 improves signal peptide predictions using deep neural networks. Nat Biotech 37: 420-423
Aviram N and Schuldiner M (2017) Targeting and translocation of proteins to the endoplasmic reticulum at a glance . J Cell Sci 130: 4079-4085
Christiansen B E et al. (2000) Methods for producing polypeptides in Aspergillus mutant cells. WO200039322
Cove D J (1966) The induction and repression of nitrate reductase in the fungus Aspergillus nidulans. Biochim Biophys Acta 113: 51-56
Gressler M et al. (2015) A new high-performance heterologous fungal expression system based on regulatory elements from the Aspergillus terreus terrein gene cluster. Front Microbiol https://doi.org/10.3389/fmicb.2015.00184
Low K O et al. (2013) Optimisation of signal peptide for recombinant protein secretion in bacterial hosts. Appl Microbiol Biotechnol 97: 3811-3826
Matsui T et al. (2014) Signal peptide for producing a polypeptide. U.S. Pat. No. 8,853,381 Matsui T et al. (2015) Recombinase-mediated integration of a polynucleotide library. WO2016026938
   Mygind P H et al. (2005) Plectasin is a peptide antibiotic with therapeutic potential from a saprophytic fungus. Nature 437: 975-980
Punt et al. (2003) The role of the Aspergillus niger furin-type protease gene in processing of fungal proproteins and fusion proteins. Evidence for alternative processing of recombinant (fusion-) proteins. J Biotechnol 106: 23-32
Olsen C L (2013) Selection in fungi. U.S. Pat. No. 9,487,767
Toida J et al. (2000) Cloning and sequencing of the triacylglycerol lipase gene of Aspergillus oryzae and its expression in Escherichia coli. FEMS Microbiol Lett 189: 159-164
Vlasenko E et al., (2010) Polypeptides having xylanase activity and polynucleotides encoding same. U.S. Pat. No. 10,202,592.
Voss M et al. (2013) Mechanism, specificity, and physiology of signal peptide peptidase(SPP) and SPP-like proteases. Biochim Biophys Acta 1828: 2828-2839
Yano A. et al. (2009) Secretory expression of the non-secretory-type Lentinula edodes laccase by Aspergillus oryzae. Microbiol Res 164: 642-649
Yaver D et al. (2007) Methods for producing secreted polypeptides having biological activity. U.S. Pat. No. 8,586,330.

1-10. (canceled)

11. A recombinant filamentous fungal host cell producing one or more secreted polypeptide of interest, said cell comprising in its genome at least one nucleic acid construct comprising a first polynucleotide encoding a signal peptide operably linked in translational fusion to a second polynucleotide encoding the polypeptide of interest, wherein the first polynucleotide is heterologous to the second polynucleotide, and wherein the first polynucleotide is selected from the group consisting of:
a) a polynucleotide having at least 70% sequence identity with SEQ ID NO: 1; and
b) a polynucleotide encoding a signal peptide having at least 70% sequence identity with SEQ ID NO: 2.

12. The filamentous fungal host cell of claim 11, wherein the first polynucleotide encodes a signal peptide comprising or consisting of the amino acid sequence of SEQ ID NO: 2.

13. The filamentous fungal host cell of claim 11, wherein the first polynucleotide encodes a signal peptide consisting of the amino acid sequence of SEQ ID NO: 2 with or without its C-terminal alanine, or a peptide fragment thereof that retains the ability to direct the polypeptide into or across a cell membrane.

14. The filamentous fungal host cell of claim 11, wherein the first polynucleotide comprises or consists of SEQ ID NO: 1 with or without its 5′ gcc codon, or a subsequence thereof which encodes a signal peptide that retains the ability to direct the polypeptide into or across a cell membrane.

15. The filamentous fungal host cell of claim 11, wherein the second polynucleotide encodes a polypeptide that is native or heterologous to the filamentous fungal host cell.

16. The filamentous fungal host cell of claim 11, wherein the second polynucleotide encodes an enzyme.

17. The filamentous fungal host cell of claim 11, wherein the second polynucleotide encodes an oxidoreductase, transferase, hydrolase, lyase, isomerase, or ligase.

18. The filamentous fungal host cell of claim 11, wherein the second polynucleotide encodes an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

19. The filamentous fungal host cell of claim 16, wherein the second polynucleotide encodes a xylanase and comprises or consists of a nucleotide sequence at least 70% identical to SEQ ID NO: 7.

20. The filamentous fungal host cell of claim 16, wherein the second polynucleotide encodes a xylanase having at least 70% sequence identity to SEQ ID NO: 8.

21. The filamentous fungal host cell of claim 11, which is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

22. The filamentous fungal host cell of claim 11, which is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma Trichoderma longibrachiatum, Trichoderma reesei, or Trichoderma viride cell.

23. The filamentous fungal host cell of claim 11, which is an Aspergillus oryzae cell.

24. A method of producing one or more secreted polypeptide of interest, said method comprising cultivating a recombinant filamentous fungal host cell as defined in claim 11 under conditions conducive to the production of the polypeptide of interest.

25. The method of claim 22, further comprising the step of recovering the polypeptide of interest.
